(19) United States
(12) Patent Application Publication
(10) Pub. No.: US 2019/0139622 A1
(43) Pub. Date: May 9, 2019

(54) GRAPH NEURAL NETWORKS FOR REPRESENTING MICROORGANISMS

(71) Applicant: Zymergen, Inc., Emeryville, CA (US)

(72) Inventor: Michael Justus Osthege, Erkrath (DE)

(21) Appl. No.: 16/053,679

(22) Filed: Aug. 2, 2018

Related U.S. Application Data
(60) Provisional application No. 62/541,011, filed on Aug. 3, 2017.

(51) Int. Cl. G16B 5/20; G06N 3/08; G16B 45/00

(52) U.S. Cl. CPC G16B 5/20; G16B 45/00; G06N 3/08

(57) ABSTRACT

Methods and systems for facilitating genetic engineering research and/or projects make and/or use a graph neural network for predicting effects of perturbations to an organism. Such perturbations may modify the organism's genome (e.g., modify a gene sequence or promoter), the organism's growth conditions (e.g., medium composition, temperature, etc.), or otherwise change the organism or its environment. The graph neural network uses graph representations of an organism, such as metabolic network representations of the organism.

### CROSS-REFERENCE TO RELATED APPLICATIONS

The application claims benefit of U.S. Provisional Patent Application No. 62/541,011, filed Aug. 3, 2017, and titled “Graph Neural Networks for Representing Microorganisms,” which is incorporated herein by reference in its entirety and for all purposes.

### FIELD

This disclosure pertains to computational methods of classifying perturbations to an organism. More particularly, the disclosure pertains to neural networks trained to classify the effects of perturbations to a gene or other feature of the organism.

### BACKGROUND

Genome engineering may be implemented in many ways. Many approaches target multiple loci in a strain's genome. Starting from a candidate strain that may be wildtype or may already have a set of genetic changes compared to the wildtype, the design decision involves selection of one or more target loci and selection of the perturbation strategy.

When a locus is selected for genetic perturbation, different types of changes can be applied to the locus. The change types might be anything from a single nucleotide change to the insertion or deletion of the gene.

Due to the size of microbial genomes, many genes might not have been previously targeted, but prior biological information about such genes is often available from various public and private sources. Further information might be available about complicating characteristics such as reaction types carried out by various genes, growth conditions, etc. It would be desirable to have an accurate predictive modeling method that is capable of incorporating large amounts of information including, for example, prior biological knowledge and/or measurements obtained from building and testing strains.

### SUMMARY

This disclosure presents methods and systems for facilitating genetic engineering research and/or projects by making and/or using graph neural networks for predicting effects of perturbations to an organism. Such perturbations may modify the organism's genome (e.g., modify a gene sequence or promoter), the organism's growth conditions (e.g., medium composition, temperature, etc.), or otherwise change the organism or its environment. The graph neural network uses graph representations of an organism, such as metabolic network representations of the organism.

Certain aspects of the disclosure pertain to methods of generating a graph neural network of metabolism as a tool for predicting an impact of one or more modifications to a gene of an organism in order to create a modified organism for producing a product. Some such methods may be characterized by the following operations: (a) generating or receiving a training set comprising a plurality of training set members, each representing a different strain of the organism, and each comprising (i) information about one or more genes present in the strain of the organism, (ii) information about one or more chemical species that are reactants or products of one or more metabolic reactions facilitated by one or more gene products expressed by at least one of the one or more genes, and (iii) information about activity of the strain of the organism; (b) organizing the information about the one or more genes of the different strains of the organism, the information about the one or more chemical species, and the information about the activity of the different strains of organism into a format for training the graph neural network; and (c) training an initial graph neural network on the organized information about the one or more genes of the different strains of the organism, the information about the one or more chemical species, and the information about activity of the different strains of the organism, wherein the training produces a trained graph neural network configured to predict activity of a new strain having one or more modifications to the gene. In certain embodiments, for each training set member, the information about the one or more genes, the one or more metabolic reactions, and the one or more chemical species is provided in the form of a graph with nodes representing the one or more genes, the one or more chemical species, and the one or more metabolic reactions.

In certain embodiments, each member of the training set includes information about, not just one gene, but two or more genes present in the strain of the organism. In certain embodiments, the graph nodes include nodes representing each of the two or more genes and the metabolic reactions of the two or more genes.

In various implementations, the graph represents a metabolic network of the organism. In various implementations, the graph represents a gene-gene interaction network of the organism. In certain embodiments, the organism is a single celled organism.

In certain embodiments, the information about the one or more genes present in the strain of the organism includes information about a mutation or a promoter of the at least one of the one or more genes. In certain embodiments, the activity of the strain is a titer of the product produced by the strain.

In certain embodiments, at least some of the nodes include features. For example, the nodes representing the one or more genes may include gene ontology features. In another example, the nodes representing the one or more genes include promoter features. In still another example, the nodes representing the one or more genes include gene modification features such as identifications of mutations. In another example, a first node representing a first metabolic reaction includes a standard reaction classification feature. In another example, a first node representing a first chemical species includes a chemical structure feature (e.g., a number of carbon atoms). In yet another example, a first node includes a first number of features and a second node includes a second number of features, where the first number and the second number are different.

In some embodiments, at least some of the training set members additionally include information about one or more environmental conditions under which the strains produce the product. In some implementations, training the initial graph neural network uses the information about one or more environmental conditions under which the strains produce the product. In certain embodiments, the information about the one or more environmental conditions is not associated with any particular node of the graph.

In certain embodiments, the form of the graph includes: a first edge representing a first chemical species, from the one or more chemical species, that is a reactant for a first one of the one or more metabolic reactions, a second edge representing a second chemical species, from the one or more chemical species, that is a product of the first one of the one or more metabolic reactions, and a third edge representing that a first gene, from the one or more genes, facilitates the first one of the one or more metabolic reactions.

In certain embodiments, the format for training the graph neural network includes one or more matrices representing the plurality of training set members. For example, the one or more matrices may include a matrix of features for the nodes representing the one or more genes. Further, the one or more matrices may include an adjacency matrix representing edges connecting at least some of the nodes representing the one or more genes, the one or more chemical species, and the one or more metabolic reactions.

In certain embodiments, the methods additionally include (d) using the trained graph neural network to predict that a new strain of the organism having a first modification of the gene will have an activity that is greater than a threshold level; and (e) making the new strain of the organism. The methods may include an additional operation of producing the product from the new strain of the organism.

Certain aspects of the disclosure pertain to methods of predicting the impact of a modification to a gene of an organism in order to create a modified organism for producing a product. Some such methods may be characterized by the following operations: (a) generating or receiving data comprising selection of a modification to a gene of the organism; (b) providing said data to a graph neural network comprising a plurality of neural network nodes having computational properties produced by training the graph neural network with (i) information about a plurality of modifications to the gene of the organism, (ii) information about one or more chemical species that are reactants or products of one or more metabolic reactions facilitated by a gene product expressed by the gene, and (iii) information about activity of the organism; and (c) predicting an activity of the organism harboring the modification to the gene. In certain embodiments, the information about the plurality of gene modifications and the one or more chemical species was provided in the form of a graph. In certain embodiments, the graph neural network was trained using information about a plurality of gene modifications to a second gene of the organism.

Depending on the prediction in (c), the method may additionally include the following operations: (d) making the organism harboring the modification to the gene; and (e) producing the product from the mutant organism. In certain embodiments, making the organism harboring the modification to the gene includes applying a mutation to the gene. In certain embodiments, applying the mutation comprises using a gene editing tool. As examples, the gene editing tool may be a TALEN system, a zinc finger system, or a CRISPR/Cas9 system designed to apply the mutation to the gene.

In certain embodiments, the information about the plurality of modifications to the gene of the organism, and the information about a one or more chemical species were provided in the form of graphs for different strains of the organism, where each graph has nodes representing the gene, the one or more chemical species, and the one or more metabolic reactions.

In various implementations, the graphs represent a metabolic network of the organism. In various implementations, the graphs represent a gene-gene interaction network of the organism. In certain embodiments, each of the graphs include: a first edge representing a first chemical species, from the one or more chemical species, that is a reactant for a first one of the one or more metabolic reactions, a second edge representing a second chemical species, from the one or more chemical species, that is a product of the first one of the one or more metabolic reactions, and a third edge representing that the gene produces a gene product that facilitates the first one of the one or more metabolic reactions.

In certain embodiments, at least some of the nodes include features. For example, the node representing the gene includes gene ontology features. In another example, the node representing the gene includes promoter features. In still another example, the node representing the gene includes gene mutation features. In another example, a first node representing a first metabolic reaction includes a standard reaction classification feature. In another example, a first node representing a first chemical species includes a chemical structure feature. In yet another example, a first node includes a first number of features and a second node includes a second number of features, where the first number and the second number are different.

In certain embodiments, the organism is a single celled organism. In certain embodiments, the activity of the organism is a titer of the product produced by the organism.

In certain embodiments, the data additionally includes information about one or more environmental conditions under which the organism produces the product. In such embodiments, predicting the activity of the organism may account for the one or more environmental conditions.

In certain embodiments, providing the data to the graph neural network includes providing one or more matrices containing features of the modified organism. For example, the one or more matrices may include a matrix of features for nodes representing one or more genes of the modified organism. Further, the one or more matrices may include an adjacency matrix representing edges connecting at least some of the nodes of the modified organism.

Certain aspects of the disclosure pertain to systems for predicting the impact of one or more modification to a gene of an organism in order to create a modified organism for producing a product. Some such systems may be characterized by a computing device comprising one or more processors and memory configured to: (i) generate or receive data comprising selection of a modification to a gene of the organism, and (ii) provide said data to a graph neural network comprising a plurality of neural network nodes having computational properties produced by training the graph neural network with:
information about a plurality of modifications to the gene of the organism,
information about one or more chemical species that are reactants or products of one or more metabolic reactions facilitated by a gene product expressed by the gene, and
information about activity of the organism, wherein the information about the plurality of gene modifications and the one or more chemical species was provided in the form of a graph.

Additionally, the computing device may be configured to predict an activity of the organism harboring the modification to the gene.

In certain embodiments, the systems additionally include a genetic engineering tool configured to produce the modified organism having the modification to the gene. In certain embodiments, the systems additionally include a bioreactor configured to produce the product from the modified organism.

In certain embodiments, the genetic engineering tool configured to produce the modified organism is configured to apply a mutation to the gene. In certain embodiments, the genetic engineering is a gene editing tool. As examples, the gene editing tool may be a TALEN system, a zinc finger system, or a CRISPR/Cas9 system designed to apply the mutation to the gene.

Training of the graph neural network may, in some implementations, involve using information about, not just a gene modification to a single gene, but about a plurality of gene modifications to a second gene of the organism. In certain embodiments, the information about the plurality of modifications to the gene of the organism, and the information about a one or more chemical species was provided in the form of graphs for different strains of the organism, each graph having nodes representing the gene, the one or more chemical species, and the one or more metabolic reactions. In various implementations, the graphs represent a metabolic network of the organism. In various implementations, the graphs represent a gene-gene interaction network of the organism.

In certain embodiments, each of the graphs include: a first edge representing a first chemical species, from the one or more chemical species, that is a reactant for a first one of the one or more metabolic reactions, a second edge representing a second chemical species, from the one or more chemical species, that is a product of the first one of the one or more metabolic reactions, and a third edge representing that the gene produces a gene product that facilitates the first one of the one or more metabolic reactions.

In certain embodiments, at least some of the nodes include features. For example, the node representing the gene includes gene ontology features. In another example, the node representing the gene includes promoter features. In still another example, the node representing the gene includes gene mutation features and/or gene promotor features. In another example, a first node representing a first metabolic reaction includes a standard reaction classification feature. In another example, a first node representing a first chemical species includes a chemical structure feature. In yet another example, a first node includes a first number of features and a second node includes a second number of features, where the first number and the second number are different.

In certain embodiments, the data additionally includes information about one or more environmental conditions under which the organism produces the product, and wherein predicting the activity of the organism accounts for the one or more environmental conditions.

In certain embodiments, the organism is a single celled organism. In certain embodiments, the activity of the organism is a titer of the product produced by the organism.

In certain embodiments, the computing device is configured to provide the data to the graph neural network by providing one or more matrices containing features of the modified organism. For example, the one or more matrices may include a matrix of features for nodes representing one or more genes of the modified organism. Further, the one or more matrices may include an adjacency matrix representing edges connecting at least some of the nodes of the modified organism.

These and other features of the disclosure will be presented below with reference to the associated drawings.

### BRIEF DESCRIPTION OF THE DRAWINGS

FIG. 1A illustrates a graph for simple example of a network having three node types.

FIG. 1B illustrates feature matrices generated for each of three different node types in a network having such node types.

FIG. 1C illustrates adjacency matrices generated for three different node types in a graph as depicted in FIG. 1A.

FIG. 2 illustrates, in symbolic fashion, a simple training set for a graph neural network. The graphs on the left would be converted to data in matrix form in order to train the neural network.

FIG. 3A presents a flow chart of a high level procedure for producing a graph neural network.

FIG. 3B presents a flow chart of a high-level procedure for using a graph neural network to, for example, assist in making a modified organism having an improved property.

FIG. 4 illustrates the matrix form of inputs to produce hidden features from graph data. A feature matrix X and a normalized adjacency matrix Â capture features and structure of a complex graph (shown on the left), and a parameters matrix Θ provides weights for calculating hidden features for the next layer of a graph neural network. Note that the information in the features matrix and the adjacency matrix is chosen for only a subset of the node types in the graph. This choice dictates the feature type of the hidden features for the next layer.

FIG. 5 illustrates an example of the matrix products for information flow in a graph neural network layer with multiple types of nodes. The graph convolution operation shown in FIG. 4 can be defined for any relationship in the graph such that all resulting hidden feature matrices Z for a particular node type can be merged.

FIG. 6A illustrates correspondence between a graph structure and a graph neural network architecture. Connections between neurons/nodes in adjacent layers of a neural network reflect connections between nodes in a corresponding graph.

FIG. 6B illustrates a multi-dimensional graph neural network volume resulting from layers having two (or more) types of nodes.

FIG. 7 illustrates a data with internal biases (only particular genes were mutated in the training set) and options for a subsequent round of genome engineering.

FIG. 8 presents an example of a computer architecture that may be employed to collect data for training sets or as inputs to neural networks, train graph neural networks, use graph neural networks to predict the impact of a modification, compare modifications, control operations associated with genome engineering (e.g., high throughput genome modifications, microorganism growth conditions, assaying), and the like.

### DETAILED DESCRIPTION

### Terminology

Terms used in the claims and specification are defined as set forth below unless otherwise specified.

The term “metabolic network” represents a set of metabolic and physical processes that influence or determine the physiological and biochemical properties of a cell. In some cases, a metabolic network includes the complete set of metabolic processes of a cell. Generally, a metabolic network includes the chemical reactions of metabolism, the metabolic pathways, and may include regulatory interactions that guide these reactions. A metabolic pathway is a linked series of chemical reactions occurring within a cell. The reactants, products, and intermediates of an enzymatic reaction are known as metabolites, which are modified by a sequence of chemical reactions catalyzed by enzymes.

The term “graph” refers to a logical structure containing a set of objects in which some pairs of the objects are in some sense “related”. The objects correspond to mathematical abstractions called nodes (also called vertices or points) and each of the related pairs of vertices is called an edge (also called an arc or line). Typically, a graph is depicted in diagrammatic form as a set of dots for the nodes, joined by lines or curves for the edges.

The term “neural network” or “artificial neural network” refers to a collection of connected units called artificial neurons or nodes. A neural network executes a series of computational operations. Each connection between artificial neurons can transmit a signal to another artificial neuron. The receiving neuron processes the signal into a value that is then used as input to downstream neurons. Neurons may have state, generally represented by real numbers, e.g., between 0 and 1. Neurons and their connections may also have a weight that varies as learning proceeds; the weights can increase or decrease the strength of the signal that it sends downstream. Further, neurons may have a threshold such that only if the aggregate signal is below (or above) that level is the downstream signal sent. Convolutional neural networks are neural networks containing one or more convolutional layers.

As used with reference to polypeptides, the term “wild-type” refers to any polypeptide having an amino acid sequence present in a polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized. The term wild-type is also used to denote naturally occurring cells.

Enzymes are identified herein by the reactions they catalyze and, unless otherwise indicated, refer to any polypeptide capable of catalyzing the identified reaction. Unless otherwise indicated, enzymes may be derived from any organism and may have a naturally occurring or mutated amino acid sequence. As is known in the art, enzymes may have multiple functions and/or multiple names, sometimes depending on the source organism from which they derive. The enzyme names used herein encompass orthologs, including enzymes that may have one or more additional functions or a different name.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST, e.g., set to default parameters.

### Introduction

Aspects of this disclosure pertain to models for predicting strain design ranking. In some embodiments, such models are trainable in a supervised fashion and support the incorporation of prior biological knowledge in form of a metabolic model, gene ontology annotations, and/or other types of prior biological metadata.

Aspects of this disclosure provide graph neural networks (GNN) that use graph representations of organisms (e.g., microorganisms) as training sets for supervised training and as inputs to trained graph neural networks. As discussed here, graph neural networks can be applied to complex property graphs with multiple types of nodes and edges such as metabolic networks. In some embodiments, approximation of spectral graph convolutions allows construction of graph (convolutional) neural networks with a computation complexity linear to the number of edges in the graph. See “Semi-Supervised Classification with Graph Convolutional Networks”, Kipf & Welling, published as a conference paper at the ICLR (2017), and “Learning Convolutional Neural Networks for Graphs,”, M. Niepert, M. Ahmed, K. Kutzkow, Proceedings of the 33^{rd} International Conference on Machine Learning (2016), incorporated herein by reference in their entireties.

In some implementations, strains are represented as instances of a metabolic network graph that may have feature-annotations on gene-nodes that describe prior biological information such as gene ontology terms (GO terms) and/or properties unique to the strain that is represented. Some such properties are unique to graph nodes (e.g., gene features indicating the type of genetic perturbation). Other properties may be provided at the graph level (not specific to any node or edge of the graph). For example, properties may indicate the presence of certain genetic features that are not tied to individual gene nodes. A productivity measurement (e.g., product titer) may be a label at graph-level and can be used for training the model.

Before proceeding, the concepts of a graph representation of an organism and perturbations to the organism will be discussed. In principal, the graphs and graph neural networks described herein can be applied to any organism including single celled organisms and higher organisms. In certain embodiments, the organism is a microbe that is naturally incapable of or has limited capability for production of the molecule of interest. In some embodiments, the microbe is one that is readily cultured, such as, for example, a microbe known to be useful as a host cell in fermentative production of molecules of interest. Bacteria, including gram positive or gram negative bacteria can be engineered. For convenience, further discussion will focus on microorganisms, and particularly strains of a single species. Often such species will be used for industrial production of particular chemical compounds.

The graph neural network may be used to identify genetic modifications or other perturbations an organism with a goal of improving the organism in some way. Examples of such perturbations include genetic modification such as nucleic acid sequence modifications (e.g., single nucleotide polymorphism changes, insertions, and/or deletions), protein sequence modifications, promoter swaps or similar modifications. Other examples include changes in the growth environment of the organism.

Generally, the graph includes a biochemical and/or genetic representation of an organism. As explained, the graphs can have nodes (sometimes referred to in the art as vertices or points) and edges connecting the nodes. The nodes represent biochemical or genetic aspects that are relevant to a property of interest for an organism (e.g., the organism's ability to produce a chemical compound in an industrial setting). Any given edge in the graph connects any two nodes. Edges may be directed or undirected and are relevant to the biochemical or genetic aspects of the nodes. They may represent different types of relationships. For example, an edge may connect an enzymatic reaction (first node) to a chemical product of the reaction (second node). If the reaction is generally irreversible, the edge is directed.

In certain embodiments, the graph includes two or more types of nodes (e.g., genes and reactions or reactions and chemical reactants). In certain embodiments, the graph includes two or more types of edge (e.g., reactions from genes and chemical products from reactions). Further examples will be provided elsewhere herein. Complex graphs, such as these, that have different types of nodes and/or edges are sometimes referred to in the art as knowledge graphs.

Examples of biological systems or networks represented by graphs include metabolic networks and gene interaction networks of an organism. The nodes and edges of the graph are typically fixed from strain-to-strain, but the features of the nodes may vary. This provides a consistent representation for capturing strain perturbations represented as feature values. These perturbations produce variable predictions, from strain-to-strain, when provided to a graph neural network.

In the case of a metabolic network graph, the nodes may include, for example, information about one or more genes, one or more metabolic reactions, and one or more chemical species. For example, the graph may include nodes representing the one or more genes, the one or more chemical species, and the one or more metabolic reactions. That is, there are different node types for genes, reactions facilitated (e.g., catalyzed) by gene-encoded proteins, and chemical compounds that are reactants or products of the reactions. In this example, the graph edges include reaction connections between reaction nodes and product (metabolite) nodes, reaction connections between reactant (metabolite) nodes and reaction nodes, and gene connections between gene nodes and reaction nodes. Additional biological information may be incorporated in form of additional relationship types (e.g., activator/repressor relationships between gene nodes).

Some or all of the nodes may be provided with associated features, some of which may represent perturbations to a base strain and some of which may represent common properties of a node (regardless of perturbation). Examples of features include gene structure (e.g., amino acid sequence), gene ontologies, gene promoters, gene product molecular weight, reaction types (e.g., standard reaction classifications such as the Enzyme Commission number and coenzymes required), and chemical structural features (e.g., number of atoms of different types (each of carbon, oxygen, nitrogen, sulfur, etc.) in the compound, bond types, pharmacophore features, and conformations). In one example, the features of a gene include multiple gene ontology classes (e.g., molecular function, cellular component(s) where the gene products are active, biological processes and/or pathways where the gene products are active, etc.), multiple promoter options, and multiple modification types.

The neural network is structured to allow representations of available perturbations in the input graph. Such representations may be provided as features of particular nodes and/or features of the graph itself. For example, variations in the amino acid sequence encoded by a gene may be provided as feature values of the gene's node. In fact, some or all amino acids in a protein may be provided as features having values representing amino acid types and/or positions in the protein. In such cases, a particular perturbation in a gene sequence may be represented as an insertion, a deletion, and/or a substitution of an amino acid or nucleotide feature in a gene node. In some cases, features of a gene node represent properties of promoters for the gene. A perturbation may be represented as a substitution (or a selection) of a baseline promoter for another known promoter. As another example, the growth or reaction conditions in which an organism produces product may be represented as a graph-level feature (e.g., a feature not associated with any particular node or edge of the graph). Perturbations to a baseline growth or reaction condition can be represented as a new condition that is a feature of the graph itself, rather than a particular node in the graph.

An example metabolic network graph contains three types of nodes and edges G=(V_{M}, V_{R}, V_{G}, E_{M→R}, E_{R→M}, E_{G→R}) where V_{T} is a set of nodes of type T and E_{TA→TB} is the set of edges from node type T_{A} to node type T_{B}. A biological interpretation of the edges is as follows:
E_{M→R}—metabolites (chemical compounds) are reactants in reactions
E_{R→M}—metabolites (chemical compounds) are products of reactions
E_{G→R}—reactions are products of genes

All three types of nodes (metabolites, reactions, genes) can have a different number of feature dimensions (F⁰_{M}, F⁰_{R}, F⁰_{G}), wherein the superscript represents the layer number (in the neural network) and the subscript represents the node type. For example, genes can be annotated with gene ontology classes, reactions can be associated with EC numbers, and metabolites can be annotated with the number of carbon atoms.

In a microbial strain optimization setting, the microbes are specialized derivatives of some model organism for which a metabolic model exists. Before gene nodes in the graph can be annotated with the type of perturbations applied in the experimental setting, the gene nodes and genome loci may need to be aligned. A pairwise alignment of amino acid sequences can produce such a mapping with high confidence.

Some gene loci that have been targeted experimentally might not align with the gene nodes from the metabolic model. Such loci may be kept for the sake of information content. These orphan genes can either be added as disconnected nodes in the graph, or they can be kept as graph-level features that become instance-level inputs to the model.

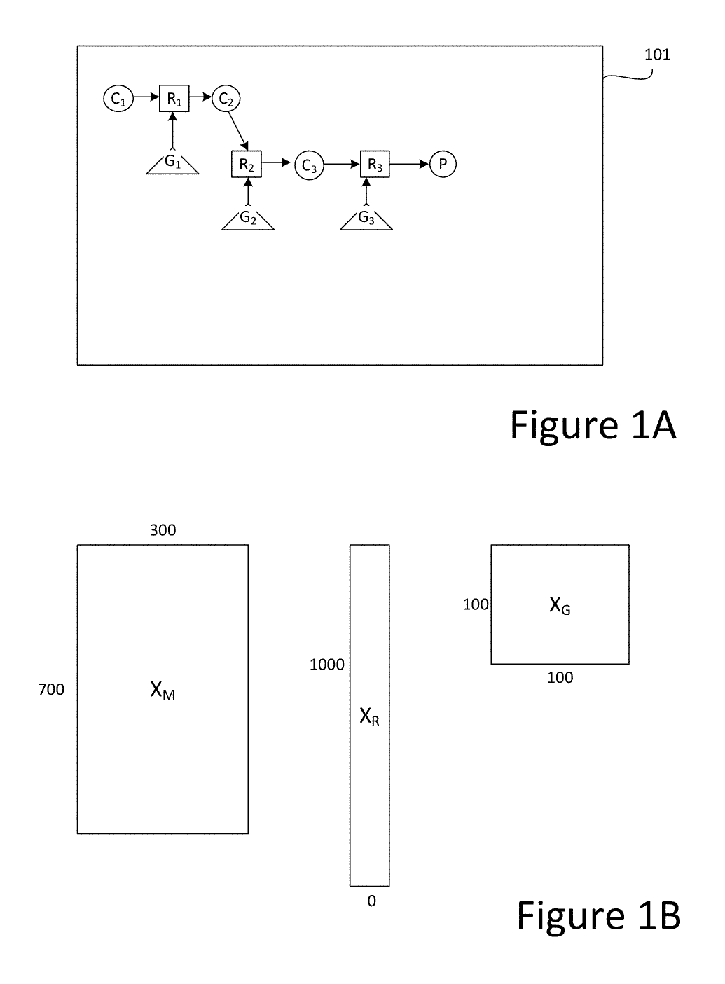

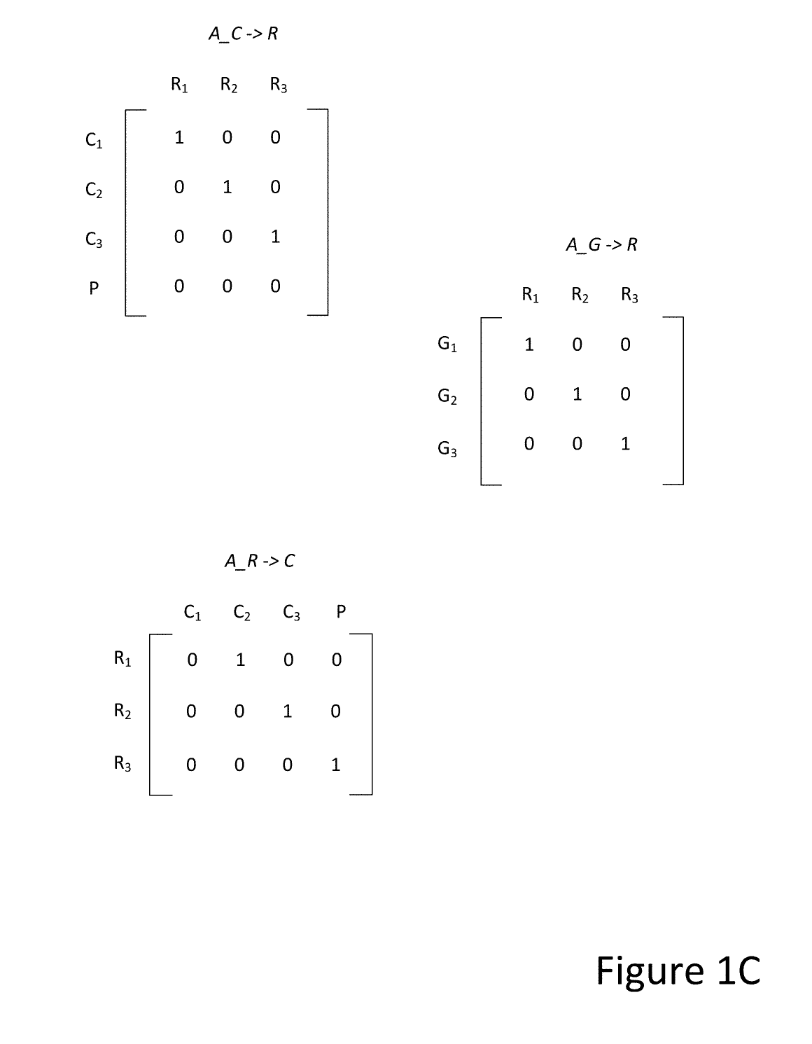

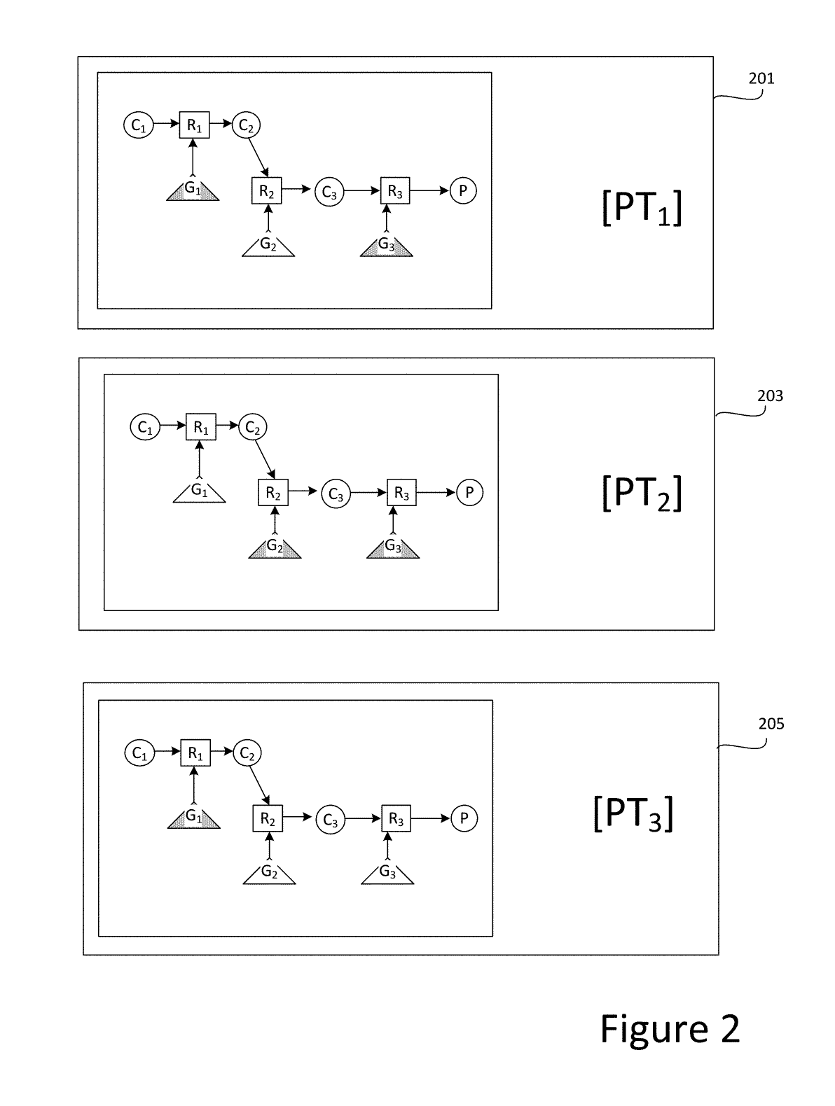

FIG. 1A shows an example of a simplified metabolic network graph. It includes three types of nodes: genes represented by triangles, reactions represented by rectangles, and chemical species (sometimes referred to as metabolites) represented by circles. It also includes three types of directed edges: genes providing polypeptides/proteins for reactions, chemical species serving as reactants for reactions, and reactions producing chemical species serving as products. Each edge is depicted as an arrow. In the depicted example, metabolism proceeds generally from left to right. A reactant chemical species (circle node C₁) participates in a first reaction (rectangle node R₁), which is enabled by a first gene (triangle node G₁), to produce a product chemical species (circle node C₂). In turn, chemical species C₂ serves as a reactant that participates in a second reaction (rectangle node R₂), which is enabled by a second gene (triangle node G₂), to produce a new product chemical species (circle node C₃). In further turn, chemical species C₃ participates in a third reaction (rectangle node R₃), which is enabled by a third gene (triangle node G₃), to produce a final product chemical species (circle node P). In a common scenario, P is the desired product of microbial production and the graph neural network is trained to predict its production. It should be understood that the depicted network is much simpler than any that would be encountered in real world organisms. Even a simple microorganism may have hundreds each of genes, reactions, and participating chemical species. As depicted in FIG. 2, metabolic network graphs may be used with other information (e.g., product titer (PT)) of organisms to define a training set for a graph neural network.

Further, while the figure shows only three types of node and three types of edges, the metabolic network is not so limited. For example, there could be other types of edges. For example, a chemical compound could be an inhibitor to an enzyme. Thus, the graph could have an enzyme inhibition edge.

As mentioned, there can be, and typically are, different numbers of features for different types of nodes. This might result in, for example, one hundred different features for gene nodes, five or fewer different features for reaction nodes, and three hundred different features for chemical species. As shown in the example of FIG. 1B, a particular organism's metabolism network graph, such as illustrated in FIG. 1A, can be can be encoded by adjacency matrices A (not shown) and feature matrices X for each type of relationship and each type of node (compounds (M), reactions (R), and genes (G)). Each of the feature matrices has rows equal to the number of nodes (of the designated type) in the network/graph and columns equal to the number of features for the node type. As an example, depicted in FIG. 1B, a first matrix X_{M} has seven hundred chemical compounds, each having three hundred features, a second matrix X_{R} one thousand reactions, each having no features, and a third matrix X_{G} has one hundred genes, each having one hundred features. This feature information can be transformed into features of a hidden layer of a graph neural network. Through a neural network structure determined by the adjacency matrices of the graph, the feature information is mathematically combined to compute hidden node features and ultimately a prediction. FIG. 1C presents adjacency matrices for each relationship (C->R, R->C, and G->R) of the simple FIG. 1A graph, where A_{C->R} has shape (N_{C} by N_{R}). As described below, the adjacency matrices of the metabolic network graph ultimately determine the structure of the neural network; in other words, the adjacency matrices mathematically incorporate the graph structure into the neural network and its predictions.

### Processes of Generating and Using Graph Neural Networks

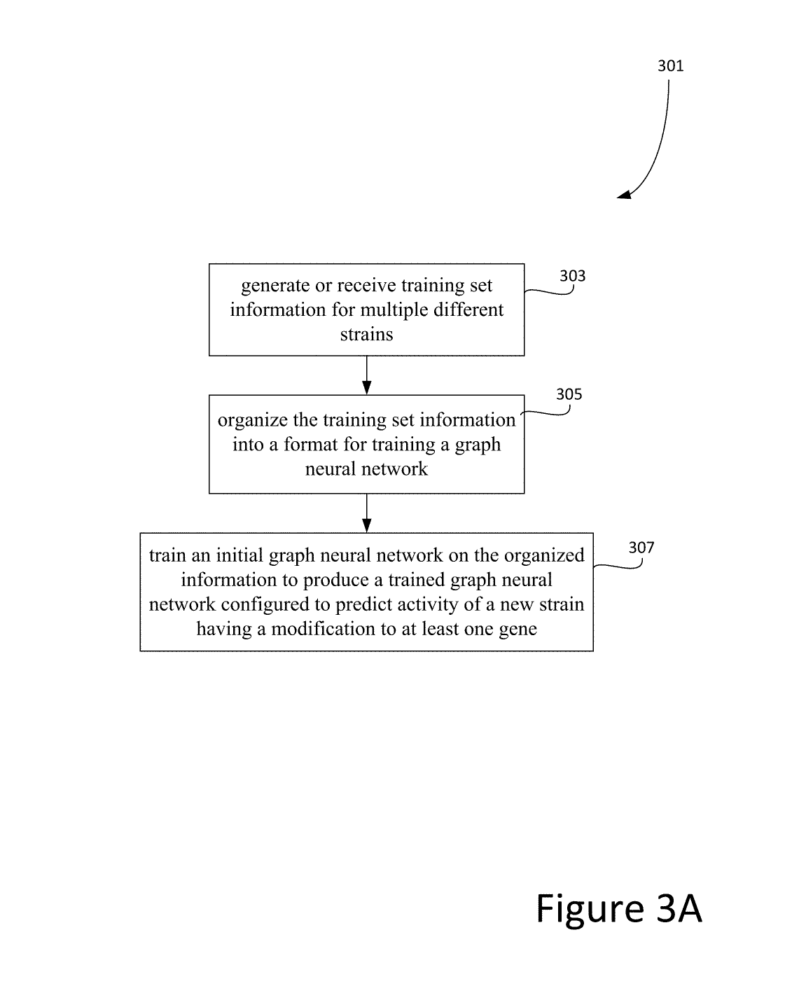

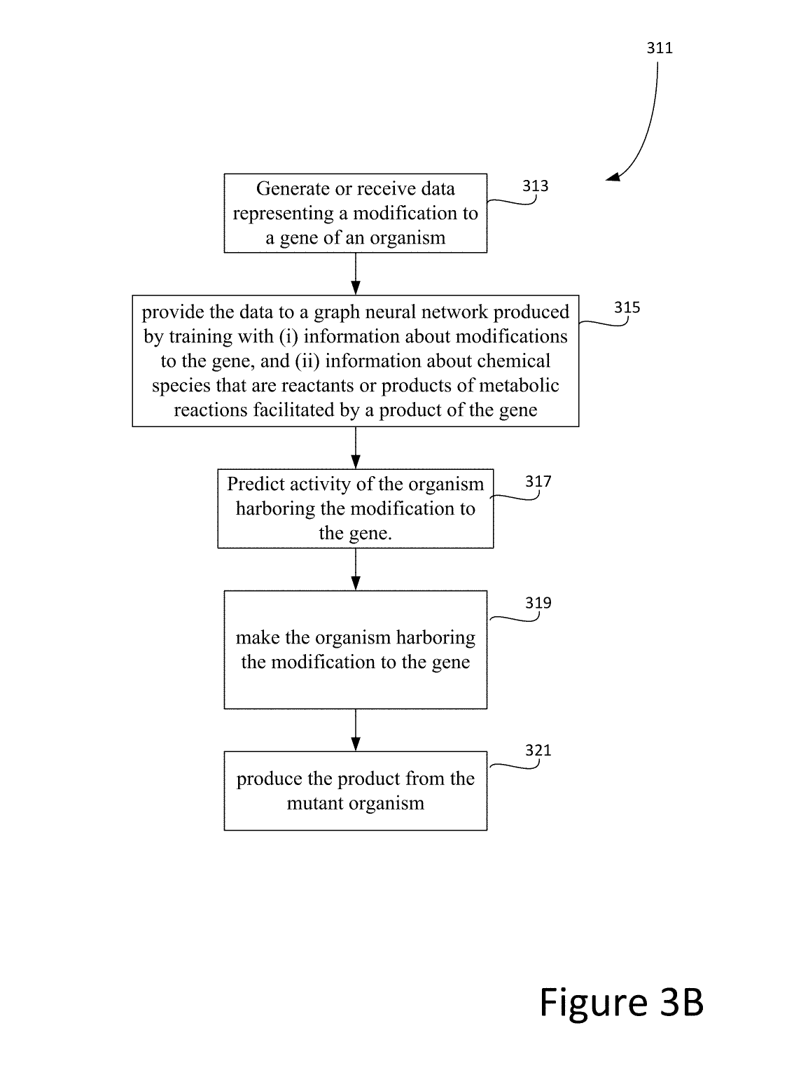

FIG. 3A is a flowchart depicting a high-level process for training a graph neural network on a biological system. The flowchart which is represented by reference number 301 begins with an operation 303 that involves generating or receiving training set information for multiple different strains of an organism. The training set information has a consistent format from strain-to-strain, but that format is multifaceted and, as explained above, can be represented as a graph. In some cases, the format includes (i) information about one or more genes present in a strain of the organism, and (ii) information about one or more chemical species that are reactants or products or intermediates of one or more metabolic reactions facilitated by one or more gene products (e.g., proteins) expressed by at least one of the genes. In the context of a metabolic network or a gene-gene interaction network, such information may be represented as different types of nodes, e.g., one type of node for genes, another type of node for chemical species, and yet another type of node for reactions. These nodes are connected by edges in a graph format. The training set data includes multiple instances of a particular graph, with each instance representing a different organism or strain of organism, differentiated by variations or modifications of genes or other features represented as nodes of the graph.

In addition to the graph components, members of the training set may include one or more pieces of graph-level information such as a level of a particular activity of an organism. In some embodiments, the activity is a result variable that is to be optimized in a genetic engineering project. For example, if the organism produces a particular product, e.g., a particular chemical compound, that is to be optimized for industrial production, the training set information may include information about an organism's ability to produce that product. For example, the training set information may include product titer or other organism activity associated with producing the desired product. Of course, other result variables may be included with the training set information. Examples include an organism's ability to live for defined a period of time under industrial production conditions. Such data may be generated by producing and testing various organisms.

FIG. 2 provides a simplified example of a training set. As illustrated, three members of a training set, member 201, member 203, and member 205, each contain the same format. That is, they include a graph representation of a strain and an activity of the strain. In this case, the activity is product titer as indicated as PT₁, PT₂, and PT₃ in the respective training set members. These activity levels are one point of distinction between the individual members the training set. In addition, the graphs have variations from member-to-member. For example, in member 201, genes G₁ and G₃ are modified; in member 203, genes G₂ and G₃ are modified; and in member 205, only gene G₁ is modified.

After generating or receiving the training set information, the process next organizes that information into a format that is suitable for training a graph neural network. See operation 305. The graph representation itself is typically not suitable for training a neural network. Instead, the information contained in the graph should be converted to matrices, vectors, or other mathematical constructs suitable for providing the data of the graph, while preserving the structural information about the graph (e.g., node types and connections between nodes). The matrices or other converted information is provided to an appropriate algorithm for training a graph neural network. As explained in more detail below, the format for training the graph neural network may include such features as adjacency matrices which represent structural information about graphs, and feature matrices which include lists of features for the various nodes in the graph.

Before training, it may also be necessary to define the architecture of the neural network, including the number and types of layers in the neural network, the nature and number of connections between the individual layers, the activation function(s), and the like. Collectively, these types of features define the graph neural network's architecture. The actual architecture for any given graph role network may be easily constructed using principles known to those of skill in the art.

After the training set information is organized into an appropriate format, it is used to train a graph neural network that is configured to predict an activity of a new strain having a putative modification to at least one gene. See block 307. In certain embodiments, the initial graph neural network contains a random set of parameters to be trained or optimized during the training process. These parameters may include weights for connections between nodes or other components of the individual layers of the neural network or other modifiable aspects of the neural network architecture. Training may be conducted using any suitable optimization algorithm such as a stochastic gradient descent technique (e.g., “Adam: A Method for Stochastic Optimization,” Kingma &. Ba, published as a conference paper at ICLR 2015, which is incorporated herein by reference in its entirety).

As mentioned, the training set information may be in form of a graph such as a graph of an organism's metabolic network or an organism's gene-gene interaction network. Often, such networks are rather complex, including at least two genes, and often many more. Similarly, in the context of a metabolic network, the network includes at least two different types of metabolic reactions that are controlled by expression products of two or more different genes. Often, the metabolic network, and the associated graph depicting it, has multiple metabolic pathways operating in parallel and series.

Often, the nodes of a graph include features. Such features may be represented as feature values organized in matrices or vectors associated with individual nodes. As explained above, gene nodes may include features that characterize the individual genes of the graph. Such characterizing features may be presented in the form of a gene ontology (such as an ontology defined by the Gene Ontology Consortium), gene promoter choices, gene modification types (such as particular mutations or locations for mutations in a gene sequence), and the like. Reaction nodes and chemical species nodes can be similarly annotated with feature values relevant to reactions and chemical species, respectively. Further, the various node types of a graph may have different numbers of features associated with them.

FIG. 3B presents a high-level flowchart representing a method of using a graph neural network to predict activity of a modified organism. As depicted, the process starts at an operation 313 that involves generating or receiving data representing a modification to a gene of an organism under investigation. As mentioned, modifications may include mutations to a gene, a promoter change, etc. Other types of perturbation to the organism may also be applied. Putative modifications may have arisen from an ongoing research effort to improve the organism's ability to produce a particular product in an industrial setting. This research effort may involve a systematic exploration of potential modifications. A graph neural network as used herein can facilitate filtering modifications that are unlikely to be successful from those that are likely to be successful.

After receiving the data representing the modification to the gene, the process next provides the data to an appropriately trained graph neural network. See block 315. As explained elsewhere herein, such neural network may be produced by training with (i) information about modifications to the gene, not necessarily including the modification proposed operation 313, and (ii) information about chemical species that are reactants and products of metabolic reactions facilitated by a product of the gene.

Upon receiving the modification information, the graph neural network will automatically predict an activity or property of the organism harboring the modification. See block 317. Assuming that the graph neural network predicts that the modification to the gene will be beneficial, the process optionally continues by building an organism that harbors the designated modification. See block 319. With the modified organism now in hand, the process may involve actually producing the product from the mutant organism. As known to those of skill in the art, the production may be done at a small scale initially and then scaled up to industrial production levels. The product can be separated from the growth medium by appropriate processing.

If the modification involves mutating to the gene, various conventional methods may be employed such as using a gene editing tool. Examples of suitable tools include TALENs, zinc finger systems, and various CRISPR systems including CRISPR/Cas9 systems. Any of these gene editing tools can be designed to apply a specific mutation to the organism's genome. Using a gene editing tool or other suitable tool, a gene may be modified by substituting a single nucleotide, inserting one or more nucleotides or codons, and/or deleting one or more nucleotides or codons.

Note that in some embodiments, not only does the process involve providing a putative modification of a gene to the graph neural network, but it may also involve some other variation on a baseline process. For example, it may specify a change to the growth conditions in which the organism produces a product. In such cases, the graph neural network is trained to account for variations in growth conditions.

Further, note that there are at least two ways a model can make a prediction or classification for a test organism: (1) the model can make predictions or classifications of nodes in a graph, or (2) the model can make predictions or classification of a property of the whole organism (e.g., product titer). Most of the discussion herein focuses on models designed to predict a property or activity of the whole organism (e.g., product titer), rather than classifying a particular node in the graph.

The development and use of graph neural networks such as exemplified in FIGS. 3A and 3B may be performed iteratively. Putative modifications that appear interesting using graph neural networks as illustrated in FIG. 3B may be applied to organisms and the resulting new organisms may be tested for titer or other result variable. The data produced in this manner may be added to a training set to refine a graph neural network or produce an entirely new graph neural network (such as produced via FIG. 3A).

### Graph Neural Network Architectures

Graph neural networks have a structure that fits certain aspects of the strain representations (e.g., graphs of metabolic networks). Specifically, graph neural networks can account for the different types of nodes and their relationships as well as their features. The features and relationships are merged for treatment in a graph neural network. Graph neural networks suitable for strain property predictions may have aspects in common with graph neural networks recently described in Kipf & Welling, “Semi-Supervised Classification with Graph Convolutional Networks,” published as a conference paper at the ICLR (2017), previously incorporated herein by reference in its entirety.

The graph neural network structure described by Kipf & Welling approximates spectral graph convolutions with a receptive field size of 1 (k=1) which results in computational complexity that is linear with respect to the number of edges in the graph. (The receptive field size is also referred to in the convolutional neural network art as the kernel size or filter size of a convolutional layer.) Architectures of graph neural networks described herein are somewhat analogous to architectures of message passing neural networks (in contrast to image convolution neural networks). As such, information flows between different types of nodes. Unlike the graph neural network by Kipf & Welling, the graph neural networks herein employ multiple types of nodes and/or different numbers of features per node type.

The computation of layers in such neural networks scales linearly with the number of the edges in the graph when implemented with sparse tensors. Property graphs, such as those employed by the neural networks described herein, can be generalized to relational graphs (G=(V, E)) where multiple types of relationships (edges) exist between nodes (vertices). Work published to date assumes that all nodes have the same number of feature dimensions. The graph neural networks disclosed herein can employ relational data with multiple types of edges and labeled nodes with different numbers of feature dimensions.

In certain embodiments, the mathematical formulation of matrix products allows the model to merge information flow from different node types. For example, the graph convolution operation can employ information flow between two types of nodes (e.g., ▴,●) with input feature dimensions F⁰● and hidden feature dimensions F¹▴. Graph convolution operations can similarly be defined for all other node and relationship types in a graph. The outputs (hidden features Z) of those operations can then be merged since the size of matrices Z is determined by the number of nodes for which the hidden features are computed and a hyperparameter F¹ that can be the same for all types of relationships.

As with most neural network architectures, the graph neural network architecture is defined by hyperparameters, which include the number of hidden layers, the number of nodes (neurons) in each hidden layer, and the activation functions of the hidden layers (or nodes in the hidden layer). Common activation functions in include sigmoid, tanh (hyperbolic tangent), ReLUs (rectified linear units), and ELU (exponential linear units).

Conventionally, a hidden feature, h, is calculated at a node using the expression

h=σ(Σ×ᵢθᵢ)+b)

where σ is the activation function, the xᵢ are input feature values, the θᵢ are learned weights, and b is a learned bias parameter. There is a separate combination of x and θ for each input connection to a node. The products xᵢθᵢ are summed over all input connections i for a node. With complex input data sets such as graphs having nodes with large numbers of features, the x and θ may be provided as matrices, X and Θ. That is, X is a feature matrix, and Θ is a parameter matrix, with h=σ(XΘ+B) where B is a matrix of bias parameters with the same shape as the result of the matrix product XΘ. In X, the number of rows equals the number of nodes of the given type and number of columns equals the number of features for the node type. In Θ, the number of rows equals the numbers of features for the node type (of X), and the number of columns equals the number of hidden features in the first hidden layer (a hyperparameter set for the architecture). The values of the parameters in Θ and B are learned during training. The variables in X are either observed (input) features or outputs of a previous layer (hidden features) that are computed from observed features and parameters.

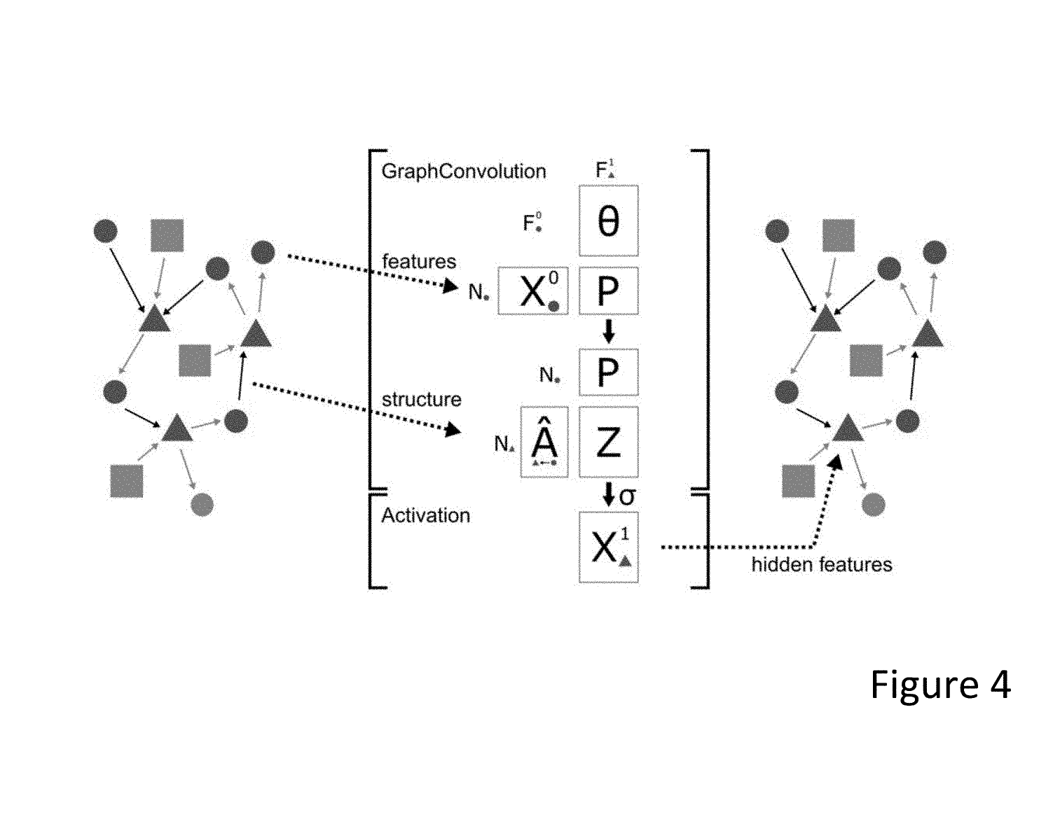

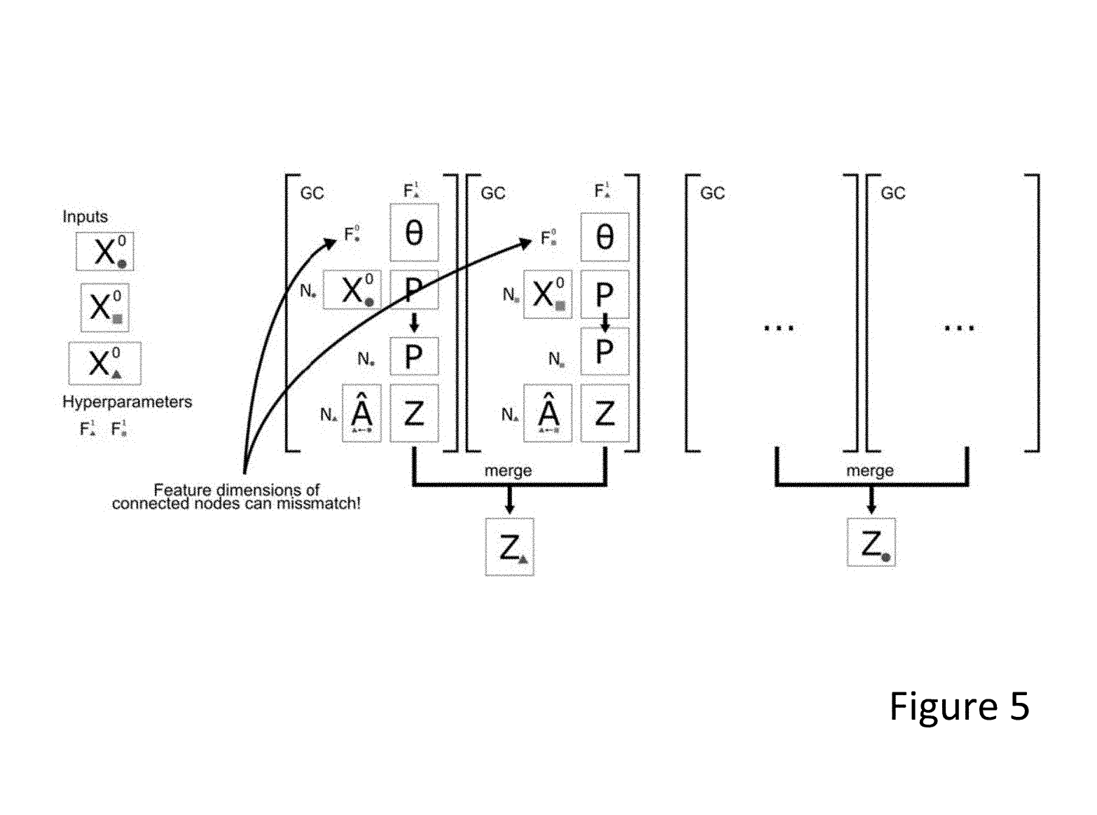

As should be apparent, the matrix Θ has dimensions (columns in the examples of FIGS. 4 and 5) set by the graph neural network architecture (specifically, the numbers of hidden and input features for the node types in question). It is equivalent to first layer hyperparameters F¹ shown in FIGS. 4 and 5.

Because there are multiple types of nodes, graph convolutional layers may be constructed using information for pairs of node types (e.g., genes and reactions), while essentially ignoring nodes of the third type. Thus, the product XΘ is calculated only for one type of node at a time. In some implementations, as illustrated in FIGS. 4 and 5, a feature matrix X₁ and a parameter matrix Θ₁, each for same first node type, are multiplied to produce a matrix of pre-combined hidden features P, which is then multiplied with a normalized adjacency matrix A₁₋₂ for the first node type and a second node type. Note that the adjacency matrix has dimensions equal to the numbers of nodes of the first and second types. Note also that in this approach, as depicted in FIGS. 4 and 5, features for nodes of a first node type (provided in X₁) are used with graph information in the adjacency matrix for nodes of the first type and a second type (A₁₋₂) to produce hidden features of the second node type. The layer depicted in FIG. 4 receives input features for nodes of a circle type and generates hidden features for nodes of a triangle type.

An adjacency matrix represents connections between nodes in a graph. Matrix positions representing node combinations where no connections exist are given values of zero and matrix positions representing node combinations where connections exist are given non-zero values. For example in a three node graph in which nodes 1 and 2 connect and nodes 1 and 3 connect, but nodes 2 and 3 do not connect, the adjacency matrix will be a 3×3 matrix having values of zero at four positions. All diagonal positions will be non-zero because each node is deemed to connect to itself. Typically, an adjacency matrix is normalized so that the sum of values in a row is equal to one. For example, if there are two non-zero entries in a row, each entry is given a value of ½. In the context of a graph having multiple node types, an adjacently matrix may have first dimension (e.g., number of columns) equal to the number of nodes of a first type (e.g., compounds) and a second dimension (e.g., number of rows) equal to the number of nodes of a second type (e.g., reactions). In some implementations, adjacency matrices are the sole source of information about edges (structure) in graphs fed to the graph neural network. In some implementations, the graph neural network employs a number of adjacency matrices equal to the number of node types squared. Therefore, if there are three node types, there may be nine adjacency matrices, each describing unlabeled directed edges between two node types: e.g., A_{M-R} , A_{R-M}, A_{R-G}, A_{G-R}, A_{G-M}, A_{M-G}, A_{M-M}, A_{G-G}, and A_{R-R}. In some architectures, all of the possible adjacency matrices for a certain relationship type (such as unlabeled directed edges) are combined into a square single super-adjacency matrix. The adjacency matrix of reactions and metabolites can be viewed as defining available reactions in the metabolic network. Transposing a matrix reverses the direction. For example, the transpose of A_{M>R} is A_{R>M}. The transpose is relevant in the context of directed edges (which may provide a non-symmetric super-adjacency matrix) and can be thought of as the transformation between the A for “inbound edges” into the A for “outbound edges.”

In FIG. 4, the graph convolution layer multiplies features of ●-nodes with weights in matrix Θ into the pre-activated and pre-convolved features matrix P. Matrix P is said to be pre-activated because the activation function (sigma) has not been applied, and it is said to be pre-convolved because its features were not yet multiplied with the adjacency matrix Â. Multiplication of a normalized adjacency matrix Â_{▴←●} with matrix P averages (convolves) the features of the ●-neighbors into pre-activated features Z of ▴-nodes. Z is fed to an activation function σ to get hidden features X_{▴}. The operation depicted in FIG. 4 may be viewed as a pre-combination operation because the features only derive from one node type.

FIG. 5 shows graph convolution across node types: hidden features of Z_{▴} are merged from separate graph convolution operations on relationships with [symbol] and ●-nodes.

The architecture of a graph convolutional neural network may reflect the structure of the graph itself. For example, the number of layers in a neural network architecture may be chosen based on the number of hops to consider in a graph. In some non-limiting examples, the maximum number of hops in the graph is determined and then used to determine the number layers in the neural network. For example, if no two nodes in a metabolic network graph are separated by more than seven hops, one may design the neural network to have seven layers. Ultimately, the number of the neural network layers depends on how many hops the designer wants information to flow through. Typically, the number of layers does not exceed the maximum number of hops between two nodes in the graph. Typically, though not necessarily, the adjacency matrix does not change depending on the number of layers in the neural network.

Further, sparseness of a graph neural network may reflect the network of edges/connections in the graph. Graph nodes that are connected to only two other nodes may be reflected in the graph neural network by neurons/nodes that have the same connectivity to other neurons of the neural network. So while any neuron in a given layer can connect to every other neuron in an adjacent layer, it does not do so. It only connects with neurons that reflect its connections to nodes in the graph.

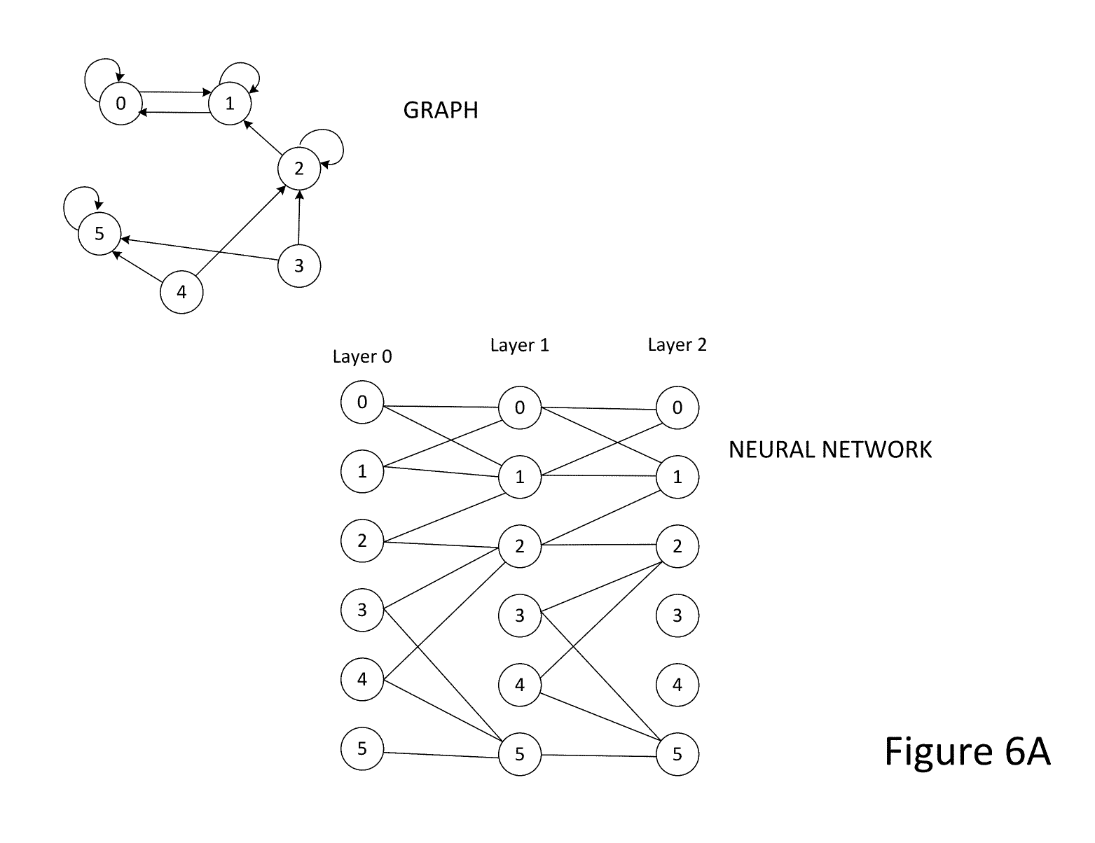

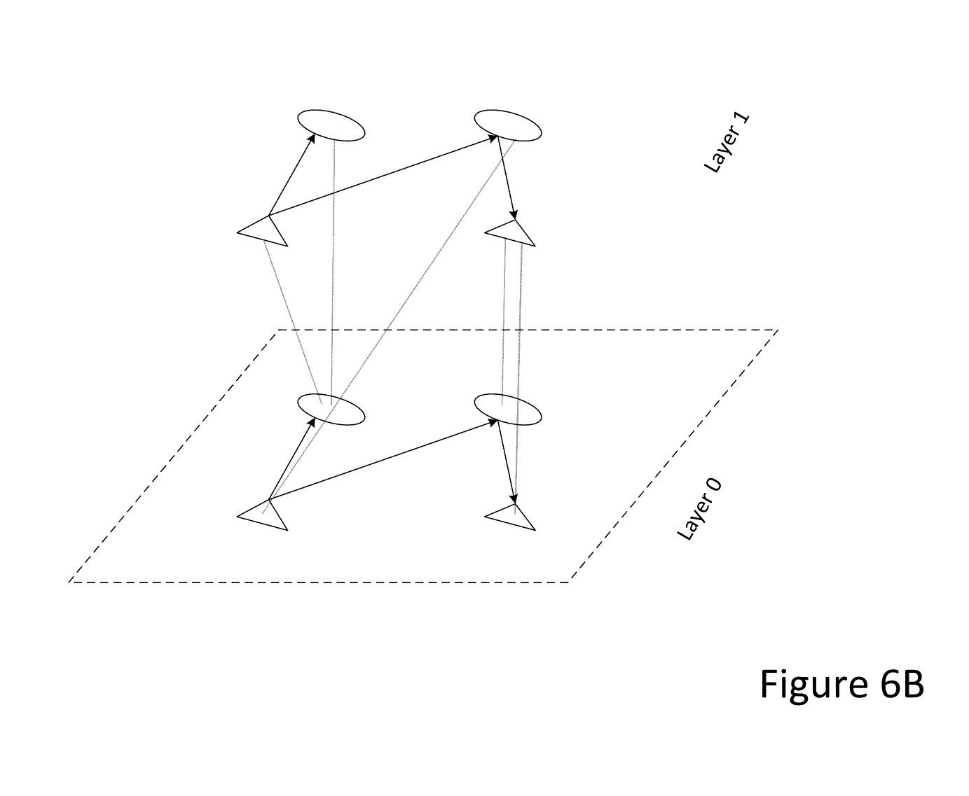

FIG. 6A helps illustrate this point by way of an example. The graph in the upper left portion of the figure contains six nodes, 0-5, with node 0 having outbound connections to itself and node 1, node 1 having outbound connections to itself and node 0, node 2 having an outbound connection to itself and node 1, node 3 having outbound connections to nodes 2 and 5, and so on. A corresponding graph neural network architecture is depicted in the lower portion of FIG. 6A. Three layers of the neural network are shown, with each layer having neurons 0-5 corresponding to nodes 0-5 of the graph. As shown in layers 0 and 1, neuron 0 has downstream connections to neurons 0 and 1 in the next layer, neuron 1 has downstream connections to neurons 0 and 1 in the next layer, neuron 2 has downstream connections to neurons 1 and 2 in the next layer, neuron 3 has downstream connections neurons 2 and 5 in the next layer, and so on. Thus, the network connections in the graph map directly to the network connections in the graph neural network. This connectivity is enforced by left-handed multiplication with the adjacency matrix in the mathematical representation discussed above. Note that any layer of a graph neural network may have one, two, or more types of nodes/neurons as dictated by the metabolic or other type of graph and the mathematical representation of the neural network. So, in practice, a graph neural network may have three or more dimensions, with one dimension being in the direction between layers and the other dimensions being directions between node/neuron types within a given layer. A simple illustration appears in FIG. 6B, which shows two layers (0 and 1), each having two types of node (circles and triangles), each with its own dimension.

After training, nodes may end up having varying importance depending on any of various factors; e.g., how close (in number of hops in a metabolic network) the node is to the final product to be produced. One may design the neural network to emphasize nodes that are deemed to be important, e.g., nodes that are close to the final product by annotating all or some nodes with the “metabolic product-proximity” that can be pre-computed from the graph structure. Or it will naturally be reflected in the weights and/or other learned parameters.

In some embodiments, a mathematical representation of the hidden features is flattened. Flattening involves converting matrices (two dimensions) to vectors (one dimension). Flattening can be accomplished by concatenating the rows (or columns) of the matrix and/or by averaging or otherwise combining values from within the matrix. The flattened representation of feature values is helpful in computing with certain architectures. For example, if information outside the graph (e.g., media properties, unconnected genes, etc.) is to be processed in the neural network, then it may be provided as a vector or scalar values that can be concatenated with matrix values that were flattened into a vector. Thus, the prediction is based on information within the graph as well as information at the level of the organism (graph-level).

In some embodiments, a single neural network architecture can be applied to multiple different metabolic networks, some with different nodes and edges. Thus, a neural network architecture developed for one microorganism can be applied to a different microorganism. With graph-level predictions this is possible if the number of hidden features that are fed to the final classification or regression layers of the neural network remains constant across organisms. This may be accomplished by averaging or otherwise combining feature values to produce a reduced number of values suitable for the applicable matrix or vector. The number of nodes in the graph however may vary since the size of the parameter matrices Θ and B only depends on the number of input/hidden feature dimensions but not the number of nodes.

### Data Splitting

In certain embodiments, some data that would otherwise be available for training models is saved for testing and validating trained models. Given that the data is sometimes limited and/or has subsets with biases, judicious choices of data splitting between training and other purposes should be made. The data splitting may be done in a way that recognizes internal biases. For example, with biases identified, the data may be split so that data with particular biases are represented in equal measure in the various data splits.

In some embodiments, there are two kinds of training: (1) fitting the selected model parameters through, e.g., backpropagation and learning to produce weights θ and biases b, and (2) determining the model hyperparameters (i.e., determining a good or the best model architecture). Therefore the available data may be split into three pools. With the first pool, the system trains the model to provide values of θ and b. Then, with the second pool, the system tests different model architectures (e.g., models with different numbers of hidden layers and/or different activation functions). Finally, after settling on a model architecture, the system validates the model with the remaining data pool.

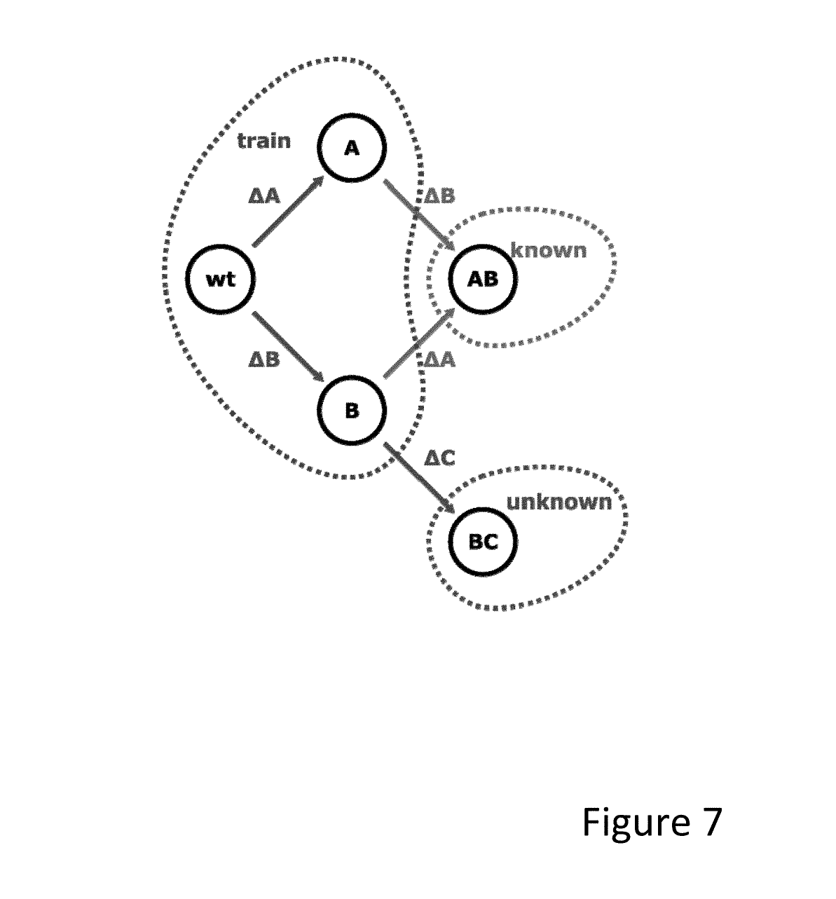

FIG. 7 illustrates a related consideration: lineage of modified organisms. A training set may include data the wild type genome as well as genomes with mutations at genes A and B. However, it does not include data on genomes with mutations at gene C, nor does it include data on genomes with mutations at both genes A and B. A model trained in this way may be more accurate in predicting activity of genomes containing mutations to only genes for which a mutation exists in the training set (e.g. mutations of genes A and B).

In a first round of engineering, genetic changes from a wild type strain result in strains A and B. In the next round, existing changes can be combined (AB) or a new change can be introduced (C). The decision on which direction to choose is impacted by the accuracy of the model in alternative directions. The accuracy is impacted by internal biases based on the training set data.

In some approaches, to get splits with known biases for the purpose of testing the model performance with respect to the particular aspects of the dataset, the data is split into:
train instances—all strains that were built before a certain date
known instances—exclusively have genetic changes that are present in at least one training strain
unknown instances—have some genetic changes that were not seen in the training data

The prediction of a model may be used as the basis for making pairwise comparisons between two organisms, or is a comparison itself. The accuracy of such pairwise comparisons is a well-interpretable metric because accuracies of 50% correspond to random decisions and any significant performance above 50% is desirable. Due to parent-child-relationships of strains, the train/known/unknown data splits still contain certain biases. When a model is asked to compare two strains from one of the two test sets, they often have different parent strains. Since the model has seen the parent strains during training, its decision would be biased by the performance of the strains parents. Therefore testing metrics may be computed that only consider pairwise comparisons between strains that share the same genetic parent. This way the model considers a specific and known difference between the strain designs and the resulting accuracy objectively informs about the model's capability in making that comparison.

### System

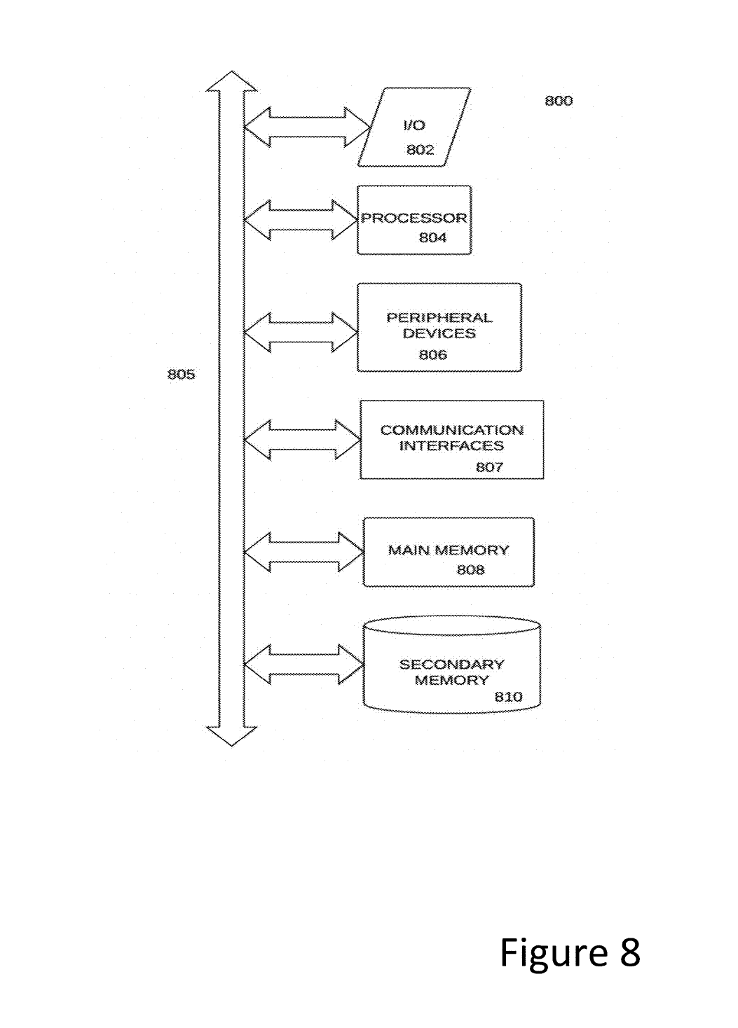

A computer system is typically used to execute program code stored in a non-transitory computer readable medium (e.g., memory) in accordance with embodiments of this disclosure. As depicted in FIG. 8, a computer system 800 may include an input/output subsystem 802, which may be used to implement an interface for interacting with human users and/or other computer systems depending upon the application. For example, the editor of embodiments of the invention may be implemented in program code on system 800 with I/O subsystem 802 used to receive input program statements from a human user (e.g., via a GUI or keyboard) and to display them back to the user. The I/O subsystem 802 may include, e.g., a keyboard, mouse, graphical user interface, touchscreen, or other interfaces for input, and, e.g., an LED or other flat screen display, or other interfaces for output. Other elements of embodiments may be implemented with a computer system like that of computer system 800, but without one or more elements such as I/O subsystem 802.

Program code may be stored in non-transitory media such as persistent storage 810 or memory 808 or both. One or more processors 804 reads program code from one or more non-transitory media and executes the code to enable the computer system to accomplish the methods performed by the embodiments herein, such as those involved with training or using a graph neural network as described herein. Those skilled in the art will understand that the processor may ingest source code, such as statements for executing training and/or modelling operations, and interpret or compile the source code into machine code that is understandable at the hardware gate level of the processor. A bus couples the I/O subsystem 802, the processor 804, peripheral devices 806, memory 808, and persistent storage 810.

Those skilled in the art will understand that some or all of the elements of embodiments of this disclosure, such as generating graphs of metabolic networks, annotating such graphs with features, producing matrices encoding graph structures and/or node features, providing such matrices to a neural network for training or predicting, and for comparing results may be implemented wholly or partially on one or more computer systems including one or more processors and one or more memory systems like those of computer system 800. Some elements and functionality may be implemented locally and others may be implemented in a distributed fashion over a network through different servers, e.g., in client-server fashion, for example.

High throughput libraries methods and systems for automating the design and construction of genetic elements to produce modified cells are known to those of skill in the art. Such techniques can be employed to produce modified microorganisms having modifications selected or identified by the neural network techniques described herein. In some implementations, these techniques employ microbial genomic engineering for controlling production of nucleotide sequences by a gene manufacturing system. In certain embodiments, a laboratory information management system is used for building, testing, and analyzing DNA sequences and/or engineered microbe genomes. Such system may integrate a microbe design phase implemented using models such as the neural networks described herein.

Some pertinent high throughput methods and apparatus are described in US Patent Application Publication US 2017/0159045, published Jun. 8, 2017, now U.S. Pat. No. 9,988,624, and International Patent Application Pub. No. WO/2017/189784, published Nov. 2, 2017, incorporated herein by reference in their entireties.

In some embodiments, a microorganism is engineered or otherwise modified based on information obtained from the graph neural network. The microorganism may be used to produce a desired molecule. A microbe that produces the molecule of interest (either naturally or via genetic engineering) can be engineered to enhance production of the molecule. In some embodiments, this is achieved by increasing the activity of one or more of the enzymes in the pathway that leads to the molecule of interest. In certain embodiments, the activity of one or more upstream pathway enzymes is increased by modulating the expression or activity of the endogenous enzyme(s). Alternatively or additionally, the activity of one or more upstream pathway enzymes can be supplemented by introducing one or more of the corresponding genes into the microbial host cell. For example, the microbe can be engineered to express multiple copies of one or more of the pathway enzymes, and/or one or more pathway enzymes can be expressed from introduced genes linked to particularly strong (constitutive or inducible) promoters. An introduced pathway gene may be heterologous or may simply be an additional copy of an endogenous gene. Where a heterologous gene is used, it may be codon-optimized for expression in the particular host microbe employed. Any or all of these modifications may be recommended using graph neural networks to evaluate many possible putative modifications.

The term “engineered” is used herein, with reference to a cell, to indicate that the cell contains at least one genetic alteration introduced by man that distinguishes the engineered cell from the naturally occurring cell.

Any microbe that can be used to express introduced genes can be engineered for fermentative production of molecules. In certain embodiments, the microbe is one that is naturally incapable fermentative production of the molecule of interest. In some embodiments, the microbe is one that is readily cultured, such as, for example, a microbe known to be useful as a host cell in fermentative production of molecules of interest. Bacteria cells, including gram positive or gram negative bacteria can be engineered as described above. Examples include C. glutamicum, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, P. citrea, Pseudomonas sp., P. alcaligenes, Lactobacilis spp. (such as L. lactis, L. plantarum), L. grayi, E. coli, E. faecium, E. gallinarum, E. casseliflavus, and/or E. faecalis cells.

The term “fermentation” is used herein to refer to a process whereby a microbial cell converts one or more substrate(s) into a desired product by means of one or more biological conversion steps, without the need for any chemical conversion step.

There are numerous types of anaerobic cells that can be used as microbial host cells in the methods described herein. In some embodiments, the microbial cells are obligate anaerobic cells. Obligate anaerobes typically do not grow well, if at all, in conditions where oxygen is present. It is to be understood that a small amount of oxygen may be present, that is, there is some level of tolerance level that obligate anaerobes have for a low level of oxygen. Obligate anaerobes engineered as described above can be grown under substantially oxygen-free conditions, wherein the amount of oxygen present is not harmful to the growth, maintenance, and/or fermentation of the anaerobes.

Alternatively, the microbial host cells used in the methods described herein can be facultative anaerobic cells. Facultative anaerobes can generate cellular ATP by aerobic respiration (e.g., utilization of the TCA cycle) if oxygen is present. However, facultative anaerobes can also grow in the absence of oxygen. Facultative anaerobes engineered as described above can be grown under substantially oxygen-free conditions, wherein the amount of oxygen present is not harmful to the growth, maintenance, and/or fermentation of the anaerobes, or can be alternatively grown in the presence of greater amounts of oxygen.

In some embodiments, the microbial host cells used in the methods described herein are filamentous fungal cells. (See, e.g., Berka & Barnett, Biotechnology Advances, (1989), 7(2):127-154). Examples include Trichoderma longibrachiatum, T. viride, T. koningii, T. harzianum, Penicillium sp., Humicola insolens, H. lanuginose, H. grisea, Chrysosporium sp., C. lucknowense, Gliocladium sp., Aspergillus sp. (such as A. oryzae, A. niger, A. sojae, A. japonicus, A. nidulans, or A. awamori), Fusarium sp. (such as F. roseum, F. graminum F. cerealis, F. oxysporuim, or F. venenatum), Neurospora sp. (such as N. crassa or Hypocrea sp.), Mucor sp. (such as M. miehei), Rhizopus sp., and Emericella sp. cells. In particular embodiments, the fungal cell engineered as described above is A. nidulans, A. awamori, A. oryzae, A. aculeatus, A. niger, A. japonicus, T reesei, T viride, F. oxysporum, or F. solani. Illustrative plasmids or plasmid components for use with such hosts include those described in U.S. Patent Pub. No. 2011/0045563.

Yeasts can also be used as the microbial host cell in the methods described herein. Examples include: Saccharomyces sp., Yarrowia sp., Schizosaccharomyces sp., Pichia sp., Candida sp, Kluyveromyces sp., and Hansenula sp. In some embodiments, the Saccharomyces sp. is S. cerevisiae (See, e.g., Romanos et al., Yeast, (1992), 8(6):423-488). In some embodiments, the Yarrowia sp. is Y. lipolytica. In some embodiments, the Kluyveromyces sp. is K. marxianus. In some embodiments, the Hansenula sp. is H. polymorpha. Illustrative plasmids or plasmid components for use with such hosts include those described in U.S. Pat. No. 7,659,097 and U.S. Patent Pub. No. 2011/0045563.

In some embodiments, the host cell can be an algal cell derived, e.g., from a green algae, red algae, a glaucophyte, a chlorarachniophyte, a euglenid, a chromista, or a dinoflagellate. (See, e.g., Saunders & Warmbrodt, “Gene Expression in Algae and Fungi, Including Yeast,” (1993), National Agricultural Library, Beltsville, Md.). Illustrative plasmids or plasmid components for use in algal cells include those described in U.S. Patent Pub. No. 2011/0045563. In other embodiments, the host cell is a cyanobacterium, such as cyanobacterium classified into any of the following groups based on morphology: Chlorococcales, Pleurocapsales, Oscillatoriales, Nostocales, or Stigonematales (See, e.g., Lindberg et al., Metab. Eng., (2010) 12(1):70-79). Illustrative plasmids or plasmid components for use in cyanobacterial cells include those described in U.S. Patent Pub. Nos. 2010/0297749 and 2009/0282545 and in Intl. Pat. Pub. No. WO 2011/034863.

Microbial cells can be engineered for using conventional techniques and apparatus of molecular biology (including recombinant techniques), microbiology, cell biology, biochemistry, and immunology, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

While many modifications suggested using neural network modelling systems described herein involve genome engineering, such modifications may be supplemented by introducing wholly new genes into microbe. Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods in Enzymology 185, Academic Press, San Diego, Calif. (1990).

In some embodiments, vectors may be used to introduce systems that can carry out genome editing, such as TALEN (transcription activator-like effector nuclease), zinc finger, meganuclease, and CRISPR systems. See U.S. Patent Pub. No. 2014/0068797, published 6 March 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F. A., et al., (“In vivo genome editing using Staphylococcus aureus Cas9,” Nature 520(7546):186-91, 2015, Apr. 9, including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Vectors or other polynucleotides can be introduced into microbial cells by any of a variety of standard methods, such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220.

The above-described methods can be used to produce engineered microbial cells that produce, and in certain embodiments, overproduce, a molecule of interest. Engineered microbial cells can have at least 1, 2, 3, 4, 5, 6, 7, 8, 9, 10, or more genetic alterations, any one or more evaluated by a neural network as described herein, as compared to a wild-type microbial cell, such as any of the microbial host cells described herein. Engineered microbial cells described in the Examples below have two genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a wild-type microbial cell. In various embodiments, engineered microbial cells can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

The engineered microbial cells can contain introduced genes that have a wild-type nucleotide sequence or that differ from wild-type. For example, the wild-type nucleotide sequence can be codon-optimized for expression in a particular host cell. The amino acid sequences encoded by any of these introduced genes can be wild-type or can differ from wild-type. In various embodiments, the amino acid sequences have at least 0 percent, 75 percent, 80 percent, 85 percent, 90 percent, 95 percent or 100 percent amino acid sequence identity with a wild-type amino acid sequence.

In various embodiments, the engineered microbial cells are capable of producing the molecule of interest at titers of at least 4, 5, 6, 7, 8, 9, 10, 20, 30, 40, 50, 75, 100, 200, 300, 400, 500, 600, 700, 800, or 900 mg/L or at least 1, 1.5, 2, 2.5, 3, 3.5, 4, 4.5, or 5 gm/L. In various embodiments, the titer is in the range of 4 mg/L to 5 gm/L, 10 mg/L to 4 gm/L, 100 mg/L to 3 gm/L, 200 mg/L to 2 gm/L, or any range bounded by any of the values listed above.

Engineered microbial cells of interest can be cultured, e.g., for maintenance, growth, and/or production of the molecule of interest. In some embodiments, the cultures are grown to an optical density at 600 nm of 10-500.

In various embodiments, the cultures produce the molecule of interest at titers of at least 50, 75, 100, 200, 300, 400, 500, 600, 700, 800, or 900 mg/L or at least 1, 1.5, 2, 2.5, 3, 3.5, 4, 4.5, or 5 gm/L. In various embodiments, the titer is in the range of 100 mg/L to 5 gm/L, 200 mg/L to 4 gm/L, 300 mg/L to 3 gm/L, or any range bounded by any of the values listed above.

The term “titer,” as used herein, refers to the mass of a product (e.g., the molecule that microbial cells have been engineered to produce) produced by a culture of microbial cells divided by the culture volume.

Microbial cells can be cultured in a minimal medium, i.e., one containing the minimum nutrients possible for cell growth. Minimal medium typically contains: (1) a carbon source for microbial growth; (2) salts, which may depend on the particular microbial cell and growing conditions; and (3) water.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include lactose and sucrose, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce the molecule of interest, the culture medium can include, and/or be supplemented during culture with, glucose and/or a nitrogen source such as urea, an ammonium salt, ammonia, or any combination thereof.

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass. Cell cultures with engineered microbial cells are often provided in a bioreactor or other production vessel, as known to those of skill in the art.

In general, cells are grown and maintained at an appropriate temperature, gas mixture, and pH (such as about 20° C. to about 37° C., about 6% to about 84% CO2, and a pH between about 5 to about 9). In some embodiments, cells are grown at 35° C. In some embodiments, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that is consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for greater than or about 1, 2, 3, 5, 10, 15, 20, 25, 30, 35, 40, 50, 60, or 70 hours. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some embodiments, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract.

The fermentation methods described herein may include an operation of recovering the molecule produced by an engineered microbial host. As used herein with respect to recovering a molecule of interest from a cell culture, “recovering” refers to separating the molecule from at least one other component of the cell culture medium. In some embodiments, the produced molecule contained in a so-called harvest stream is recovered/harvested from the production vessel. The harvest stream may include, for instance, cell-free or cell-containing aqueous solution coming from the production vessel, which contains the produced molecule. Cells still present in the harvest stream may be separated from the molecule by any operations known in the art, such as for instance filtration, centrifugation, decantation, membrane crossflow ultrafiltration or microfiltration, tangential flow ultrafiltration or microfiltration or dead end filtration. After this cell separation operation, the harvest stream is essentially free of cells.

Further steps of separation and/or purification of the produced molecule from other components contained in the harvest stream, i.e., so-called downstream processing steps may optionally be carried out. These steps may include any means known to a skilled person, such as, for instance, concentration, extraction, crystallization, precipitation, adsorption, ion exchange, chromatography, distillation, electrodialysis, bipolar membrane electrodialysis and/or reverse osmosis. Any of these procedures can be used alone or in combination to purify the produced molecule. Further purification steps can include one or more of, e.g., concentration, crystallization, precipitation, washing and drying, treatment with activated carbon, ion exchange and/or re-crystallization. The design of a suitable purification protocol may depend on the cells, the culture medium, the size of the culture, the production vessel, etc. and is within the level of skill in the art.

### Conclusion

None of the claims herein include limitations presented in “means plus function” or “step plus function” form. (See, 35 USC § 112(f)). It is Applicant's intent that none of the claim limitations be interpreted under or in accordance with 35 U.S.C. § 112(f).

While the present invention has been particularly described with respect to the illustrated embodiments, it will be appreciated that various alterations, modifications and adaptations may be made based on the present disclosure, and are intended to be within the scope of the present invention. While the invention has been described in connection with the disclosed embodiments, it is to be understood that the present invention is not limited to the disclosed embodiments but, on the contrary, is intended to cover various modifications and equivalent arrangements included within the scope of the claims.

What is claimed is:

1. A method of generating a graph neural network of metabolism as a tool for predicting an impact of one or more modifications to a gene of an organism to create a modified organism for producing a product, the method comprising:
(a) generating or receiving a training set comprising a plurality of training set members, each representing a different strain of the organism, and each comprising (i) information about one or more genes present in the strain of the organism, (ii) information about one or more chemical species that are reactants or products of one or more metabolic reactions facilitated by one or more gene products expressed by at least one of the one or more genes, and (iii) information about activity of the strain of the organism, wherein, for each training set member, the information about the one or more genes, the one or more metabolic reactions, and the one or more chemical species is provided in the form of a graph with nodes representing the one or more genes, the one or more chemical species, and the one or more metabolic reactions;
(b) organizing the information about the one or more genes of the different strains of the organism, the information about the one or more chemical species, and the information about the activity of the different strains of organism into a format for training the graph neural network; and
(c) training an initial graph neural network on the organized information about the one or more genes of the different strains of the organism, the information about the one or more chemical species, and the information about activity of the different strains of the organism, wherein the training produces a trained graph neural network configured to predict activity of a new strain having one or more modifications to the gene.

2. The method of claim 1, wherein each member of the training set comprises information about two or more genes present in the strain of the organism.

3. The method of claim 1, wherein the graph represents a metabolic network of the organism.

4. The method of claim 1, wherein the format for training the graph neural network comprises one or more matrices representing the plurality of training set members.

5. The method of claim 4, wherein the one or more matrices comprises a matrix of features for the nodes representing the one or more genes, and wherein the one or more matrices comprises an adjacency matrix representing edges connecting at least some of the nodes representing the one or more genes, the one or more chemical species, and the one or more metabolic reactions.

6. The method of claim 1, further comprising:
using the trained graph neural network to predict that a new strain of the organism having a first modification of the gene will have an activity that is greater than a threshold level; and
making the new strain of the organism.

7. The method of claim 6, further comprising producing the product from the new strain of the organism.

8. The method of claim 1, wherein the information about the one or more genes present in the strain of the organism comprises information about a mutation or a promoter of the at least one of the one or more genes.

9. The method of claim 1, wherein the form of the graph comprises: a first edge representing a first chemical species, from the one or more chemical species, that is a reactant for a first one of the one or more metabolic reactions, a second edge representing a second chemical species, from the one or more chemical species, that is a product of the first one of the one or more metabolic reactions, and a third edge representing that a first gene, from the one or more genes, facilitates the first one of the one or more metabolic reactions.

10. The method of claim 1, wherein at least some of the nodes comprise features.

11. The method of claim 1, wherein at least some of the training set members further comprise information about one or more environmental conditions under which the strains produce the product, and wherein training the initial graph neural network uses the information about one or more environmental conditions under which the strains produce the product, and wherein the information about the one or more environmental conditions is not associated with any particular node of the graph.

12. A method of predicting the impact of a modification to a gene of an organism to create a modified organism for producing a product, the method comprising:
(a) generating or receiving data comprising selection of a modification to a gene of the organism;
(b) providing said data to a graph neural network comprising a plurality of neural network nodes having computational properties produced by training the graph neural network with (i) information about a plurality of modifications to the gene of the organism, (ii) information about one or more chemical species that are reactants or products of one or more metabolic reactions facilitated by a gene product expressed by the gene, and (iii) information about activity of the organism, wherein the information about the plurality of gene modifications and the one or more chemical species was provided in the form of a graph; and
(c) predicting an activity of the organism harboring the modification to the gene.

13. The method of claim 12, further comprising:
making the organism harboring the modification to the gene; and
producing the product from the mutant organism.

14. The method of claim 13, wherein making the organism harboring the modification to the gene comprises applying a mutation to the gene.

15. The method of claim 12, wherein the information about the plurality of modifications to the gene of the organism, and the information about a one or more chemical species was provided in the form of graphs for different strains of the organism, each graph having nodes representing the gene, the one or more chemical species, and the one or more metabolic reactions.

16. The method of claim 12, wherein the organism is a single celled organism.

17. The method of claim 12, wherein the activity of the organism is a titer of the product produced by the organism.

18. A system for predicting the impact of one or more modification to a gene of an organism to create a modified organism for producing a product, the system comprising:
(a) a computing device comprising one or more processors and memory, wherein the computing device is configured to
(i) generate or receive data comprising selection of a modification to a gene of the organism,
(ii) provide said data to a graph neural network comprising a plurality of neural network nodes having computational properties produced by training the graph neural network with
information about a plurality of modifications to the gene of the organism,
information about one or more chemical species that are reactants or products of one or more metabolic reactions facilitated by a gene product expressed by the gene, and
information about activity of the organism,
wherein the information about the plurality of gene modifications and the one or more chemical species was provided in the form of a graph, and
(iii) predict an activity of the organism harboring the modification to the gene; and
(b) a genetic engineering tool configured to produce the modified organism having the modification to the gene.

19. The system of claim 18, further comprising a bioreactor configured to produce the product from the modified organism.

20. The system of claim 18, wherein the information about the plurality of modifications to the gene of the organism, and the information about a one or more chemical species was provided in the form of graphs for different strains of the organism, each graph having nodes representing the gene, the one or more chemical species, and the one or more metabolic reactions.

21. The system of claim 20, wherein each of the graphs comprise: a first edge representing a first chemical species, from the one or more chemical species, that is a reactant for a first one of the one or more metabolic reactions, a second edge representing a second chemical species, from the one or more chemical species, that is a product of the first one of the one or more metabolic reactions, and a third edge representing that the gene produces a gene product that facilitates the first one of the one or more metabolic reactions.

22. The system of claim 18, wherein the organism is a single celled organism.

23. The system of claim 18, wherein the data further comprises information about one or more environmental conditions under which the organism produces the product, and wherein predicting the activity of the organism accounts for the one or more environmental conditions.

24. The system of claim 18, wherein the genetic engineering tool configured to produce the modified organism is configured to apply a mutation to the gene.

25. The system of claim 24, wherein the genetic engineering tool configured to produce the modified organism comprises a gene editing tool.

26. The system of claim 24, wherein the gene editing tool is a TALEN system, a zinc finger system, or a CRISPR/Cas9 system designed to apply the mutation to the gene.

27. A microorganism comprising a modification to the gene where the modification is predicted to have a positive impact on the microorganism by the graph neural network produced by the method of claim 1.
